(19) United States
(12) Patent Application Publication
(10) Pub. No.: US 2025/0380859 A1
(43) Pub. Date: Dec. 18, 2025

(54) DEVICES, SYSTEMS, AND METHODS FOR ACTUATING PORTIONS OF A MEDICAL DEVICE

(71) Applicant: Boston Scientific Scimed, Inc., Maple Grove, MN (US)

(72) Inventor: Evan WILDER, Waltham, MA (US)

(73) Assignee: Boston Scientific Scimed, Inc., Maple Grove, MN (US)

(21) Appl. No.: 19/233,247

(22) Filed: Jun. 10, 2025

Related U.S. Application Data
(60) Provisional application No. 63/659,581, filed on Jun. 13, 2024.

(51) Int. Cl. A61B 1/005

(52) U.S. Cl. CPC A61B 1/0057

(57) ABSTRACT

A medical device comprising a knob with a recessed first portion and a shaft with an articulation section. A control rod is received within the first portion of the knob and the knob is configured to actuate an articulation wire. Actuation of the knob is configured to move the articulation section. The knob is configured to transition between a first state, in which the knob is fixedly coupled to the control rod, and a second state, in which the knob is moveable relative to the control rod. The knob is configured to transition from the first state to the second state when a threshold torque value is applied to the knob.

### CROSS-REFERENCE TO RELATED APPLICATIONS

This application claims the benefit of priority of U.S. Provisional Patent Application No. 63/659,581, filed on Jun. 13, 2024, the entirety of which is incorporated herein by reference.

### TECHNICAL FIELD

Aspects of this disclosure generally relate to devices, systems, and methods for actuating a portion of a medical device. Some aspects relate to medical devices, systems, and methods for actuating a portion of a medical device via a handle actuator, such as actuation via one or more articulation wires and a knob of a handle of a medical device.

### BACKGROUND

During a medical procedure, a user may operate a medical device, including a handle and a shaft extending distally from a portion of the handle. Some medical devices include at least one control knob that controls movement of a distal articulating section of the shaft. The distal articulating section's movement enables users to navigate complex anatomical structures. The distal articulating section may be coupled to one or more articulation wires, which in turn are coupled to one or more control knobs. The user may actuate a control knob, which may control articulation of the distal articulation section in various directions via the one or more articulation wires.

Conventional control knobs may have a limited range of motion and include “hard stop” features integrated within the handle to achieve this limited range of motion. The “hard stop” features in the handle may interact with the control knobs to limit articulation of the distal articulation section, for example by limiting the distal or proximal movement of one or more articulation wires. During navigation through longer and more tortuous anatomical pathways, the relative length of the articulation wires may vary. The variability in wire length can lead to biases in the control knobs towards certain hard stops, consequently further limiting the range of motion of one or more articulation wires in one direction.

The devices, systems, and methods of this disclosure may help to rectify one or more of the issues described above or address other aspects of the art.

### SUMMARY

In one example, a medical device may comprise a knob with a recessed first portion and a shaft with an articulation section. A control rod may be received within the first portion of the knob. The knob may be configured to actuate an articulation wire and actuation of the knob may be configured to move the articulation section. The knob may be configured to transition between (1) a first state, in which the knob may be fixedly coupled to the control rod, and (2) a second state, in which the knob may be movable relative to the control rod. The knob may be configured to transition from the first state to the second state when a threshold torque value is applied to the knob.

According to some aspects, the first portion may include a radially-inward facing surface, relative to a central longitudinal axis of the first portion that is polygonal shaped. The control rod may include a radially-outer facing surface, relative to a central longitudinal axis of the control rod, which aligns with the polygonal shaped radially-inward facing surface of the first portion. The first portion may also include a radially-inward facing surface, relative to a central longitudinal axis of the first portion, and the radially-inward facing surface may include a plurality of planar surfaces. The medical device may include a threshold torque value within the range of 5 lbs to 15 lbs. The control rod may be coupled to the articulation wire and the articulation wire may extend longitudinally through the shaft. The rotation of the knob may be configured to move the articulation wire proximally or distally when the knob is in the first state, and rotation of the knob may be configured to not move the articulation wire proximally or distally when the knob is in the second state.

The knob may include a plurality of semicircular components surrounding the control rod, and a radially-inward facing surface of each of the plurality of semicircular components that may align with a shape of the control rod. The semicircular components may be coupled to a separate biasing member. The biasing member(s) may be a spring, and each of the springs may be configured to bias one of the plurality of semicircular components towards a central longitudinal axis of the first portion. Upon reaching the threshold torque value, each of the biasing members may be configured to compress and increase a diameter of the first portion and when the threshold torque value has not been reached, each of the semicircular components may abut at least two other semicircular components.

Further, the control rod may include at least one first magnet, and the knob may include at least one second magnet. A positive pole of the at least one first magnet may face a direction away from a central longitudinal axis of the control rod, and a negative pole of the at least one second magnet may face towards the central longitudinal axis of the control rod. The at least one second magnet may include a plurality of second magnets, and the plurality of second magnets may be arranged circumferentially around the first portion of the knob.

The disclosure also includes a medical device comprising a knob that may include a recessed first portion. The knob may be configured to actuate an articulation wire. The first portion may be configured to receive a control rod. The knob may be configured to transition between (1) a first state in which the knob may be fixedly coupled to the control rod and (2) a second state in which the knob may be movable relative to the control rod. The knob may be configured to transition from the first state to the second state when a threshold torque value is applied to the knob.

According to some aspects, a radially-inward facing surface, relative to a central longitudinal axis of the first portion of the knob includes a plurality of planar surfaces and corresponds to a polygonal surface of the control rod. The knob may comprise at least one magnet. The knob may comprise at least one biasing member that may be coupled to a semicircular component of the first portion, and the semicircular component may be moveable relative to a plurality of prongs of the knob.

The disclosure also includes a medical device comprising a knob that may have a radially-outer portion and a recessed first portion. The knob may be configured to actuate an articulation wire, and the first portion may be configured to receive a control rod. The knob may be configured to transition between (1) a first state in which the knob is fixedly coupled to the control rod and (2) a second state in which the knob is moveable relative to the control rod. The knob may be configured to transition from the first state to the second state when a threshold torque value is applied to the knob. Furthermore, the first portion may include a plurality of semicircular components, and each of the plurality of semicircular components may be (i) coupled to a separate biasing member of a plurality of biasing members and (ii) moveable relative to the radially-outer portion of the knob. According to some aspects, the plurality of semicircular components may be configured to move away from a central longitudinal axis of the knob when the plurality of biasing members are compressed and moved radially outward.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 is a perspective view of a medical device, according to an embodiment.

FIG. 2 is a side view of an interior of a proximal portion of a medical device, according to an embodiment.

FIG. 3A is a side view of an interior of a proximal portion of a handle of a medical device.

FIG. 3B is a side view of a shaft and articulation wires of the medical device of FIG. 3A.

FIG. 4A is a side view of the interior of the handle of the medical device of FIG. 3A and FIG. 3B.

FIG. 4B is a side view of the shaft and articulation wires of the medical device of FIG. 3A, FIG. 3B and FIG. 4A.

FIG. 5 is a perspective view of a portion of a handle and a knob, according to aspects of this disclosure.

FIG. 6 is a frontal view of a portion of a handle and a knob, according to aspects of this disclosure.

FIG. 7 is a perspective view of a portion of a handle and a knob, according to aspects of this disclosure.

### DETAILED DESCRIPTION

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements but may include other elements not expressly listed or inherent to such a process, method, article, or apparatus. In this disclosure, relative terms, such as, for example, “about,” “substantially,” “generally,” and “approximately” are used to indicate a possible variation of +10% in a stated value or characteristic.

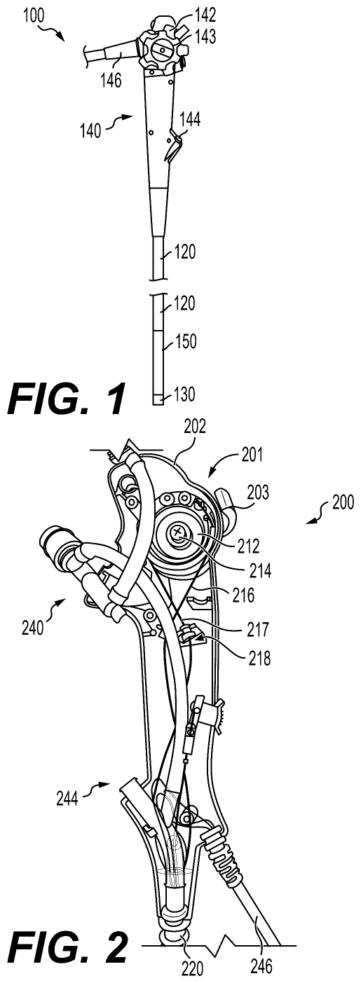

Referring to FIG. 1, a medical device 100, according to an embodiment, is shown. Medical device 100 may be an exemplary endoscope. However, any other medical device (e.g., bronchoscope, colonoscope, ureteroscope, duodenoscope, gastroscope, colonoscope, laparoscope, aspiration scope, etc.), catheter, sheath, or the like may be used in combination with aspects of this disclosure.

Medical device 100 includes a handle 140 and a shaft 120 including a tip 130 at a distalmost end of shaft 120 and an articulation section 150 at a distal portion of shaft 120. Handle 140 is coupled to a proximal end of shaft 120. Handle 140 may include a first actuator or knob 142, a second actuator or knob 143, and a port 144. The first actuator or knob 142 and/or the second actuator or knob 143 may be configured to control articulation of the articulation section 150. Port 144 of handle 140 may be configured for introducing and/or removing tools, fluids, or other materials to and from the shaft 120 for delivery to a target site and may be fluidically connected with shaft 120. In addition, an umbilicus 146 for introducing fluid, suction, and/or wiring for electronic components may be coupled to handle 140.

A plurality of actuating elements, such as cables or wires, may extend distally from a proximal end of medical device 100 (such as handle 140) to articulation section 150 and/or tip 130. For example, articulation/actuation wires may be indirectly coupled to first actuator or knob 142 and/or second actuator or knob 143, which may control articulation of articulation section 150 in multiple directions, such as up, down, left, and right (i.e., four-way steering). FIG. 2 illustrates exemplary articulation wires 216, 217 within a handle body 202 of a medical device 200, and will be discussed further below. Knobs 142, 143 may be, for example, rotate relative to handle 140 to push/pull actuating elements (e.g., articulation wires 216, 217 in FIG. 2). Medical device 100, and any medical device described herein, may include two control knobs used for four-way steering of an articulation section of a shaft.

One or more cameras or other viewing elements (not shown) may be incorporated into medical device 100, may be positioned at tip 130, and may be used for direct visualization of a patient's internal anatomy, for example the pancreaticobiliary anatomy for diagnostic or therapeutic purposes. In some examples, the one or more cameras or other viewing elements may be controlled via handle 140 by a user of medical device 100, such as via one or more actuators of handle 140. Tip 130 may also include one or more illuminators, one or more exit openings of one or more working channels, and/or one or more elevators.

FIG. 2 shows the inside of an exemplary proximal portion of a medical device 200. Medical device 200 may include any of the features of medical device 100. A distal portion of medical device 200 may be similar to the distal portion of medical device 100 and include similar components (e.g., shaft 220 being similar to shaft 120 and may include an articulation section 150 and a tip 130.) The proximal portion of medical device 200 may include a handle 240 sized and/or shaped to be held by the user of medical device 200. Note a portion of handle 240 is removed in FIG. 2 to expose the interior components of handle 240. Handle 240 may include an umbilicus 246, an actuator 203, a port 244, and a housing or a handle body 202. Shaft 220 may be coupled to a distal end of handle 240.

Various components of medical device 200 may be disposed within the handle body 202 of handle 240. A rod 214 may be coupled to handle body 202 and configured to receive a knob (e.g., knob 142). In some examples, rod 214 may be coupled to an articulation wheel 212. Handle body 202 of handle 240 may include an articulation mechanism 201 configured to move a tip (e.g., tip 130) of shaft 220 when an actuator 203 (such as a knob, lever, button, etc.) is actuated. In some examples, the articulation mechanism 201 may include a knob (not shown), rod 214, articulation wheel 212, and one or more articulation wires 216, 217.

The user of medical device 200 may cause an articulation section of shaft 220 (e.g., articulation section 150) to bend in one of at least two directions by actuating an actuator 203 in a given direction. In some examples, the user may rotate a knob coupled to rod 214 to move one or more articulation wires 216, 217 and thus bend an articulation section of shaft 220. Actuator 203 may be coupled to and/or configured to actuate articulation wheel 212, for example to rotating rod 214 to rotate articulation wheel 212. In some examples, two articulation wires 216, 217 may be coupled to articulation wheel 212. For example, articulation wires 216, 217 may be coupled around the circumference of articulation wheel 212. Articulation wheel 212 may include grooves or channels that guide articulation wires 216, 217 and ensure articulation wires 216, 217 remain in an appropriate position within handle 240. Articulation wheel 212 may also have connection points used to couple articulation wires 216, 217, such as protrusions used to couple articulation wires 216, 217 to articulation wheel 212.

Articulation wires 216, 217 may be moved in a first direction, via actuator 203 or other actuator (e.g., a knob) coupled to rod 214, by rotation of articulation wheel 212. When articulation wheel 212 is rotated in a first direction, articulation wire 216 may be moved (e.g. pulled or tensioned) proximally and articulation wire 217 may be moved (e.g. pushed or loosened) distally, and a distal end (e.g., tip 130 and/or an articulation section of shaft 220) may move in a third direction. Conversely, when actuator 203 (or a knob or other actuator coupled to rod 214) is moved in a second direction (e.g. rotated in an opposite direction from the first direction, etc.), articulation wheel 212 may rotate in the second direction and articulation wire 217 may be moved (e.g. tensioned and/or pulled) proximally and articulation wire 216 may be moved (e.g. pushed or loosened) distally, causing a distal end (e.g. tip 130) of shaft 220 to move (e.g. bend) in a fourth direction opposite from the third direction. In some examples, one or more guide elements 218 may be positioned distally from articulation wheel 212 and may be configured to receive and/or guide and/or retain articulation wires 216, 217 (e.g., to prevent entanglement and/or interference with other interior components within handle body 202).

In an exemplary construction discussed in more detail below, articulation wheel 212 (including any articulation wheel described herein) and aspects within handle body 202 may include hard stop features. Hard stop features may limit articulation of articulation wires (e.g., articulation wires 216, 217) when articulation by articulation mechanism 201 reaches a threshold torque, preventing articulation wheel 212 from rotating further and limiting movement of articulation wires 216, 217. Hard stop features may help prevent breakage or articulation wires 216, 217 during operation.

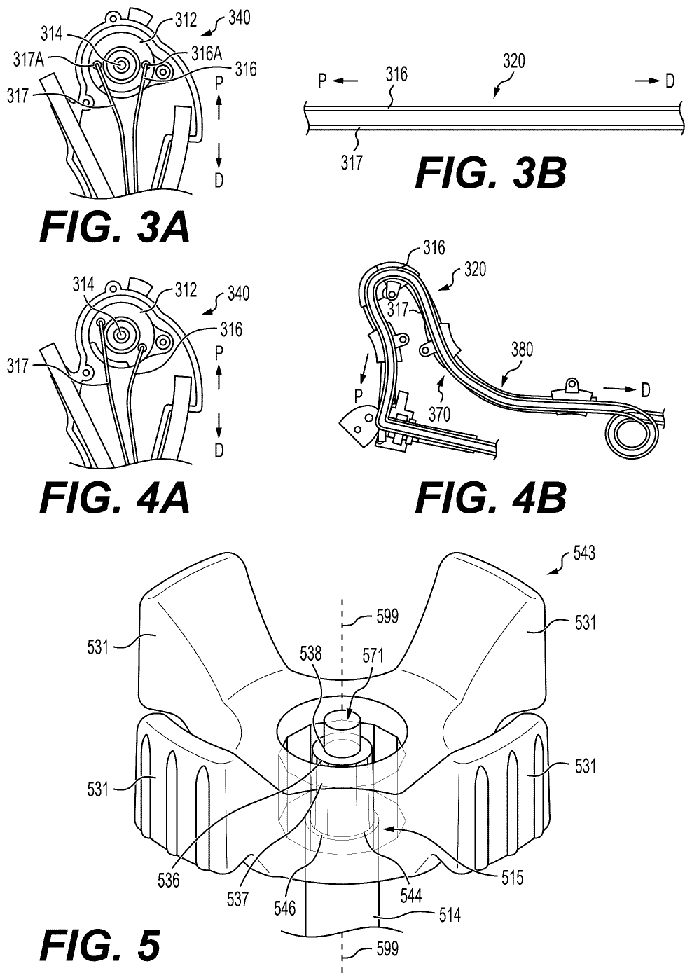

FIGS. 3A and 4A depict an exemplary handle 340 that may have any of the features of handle 140 and handle 240. Handle 340 may include an articulation wheel 312, and one or more articulation wires 316, 317 (e.g., similar to articulation wires 216, 217) coupled to articulation wheel 312. Articulation wheel 312 may surround and be fixedly coupled to rod 314. Rod 314 may be configured to receive a knob (not shown) at an exterior portion of handle 340. As discussed above, the knob may cause articulation wheel 312 to rotate at least in a first direction and/or a second direction. When the knob is rotated, articulation wires 316, 317 may be moved proximally or distally, and a distal end (e.g., tip 130) may move in a first direction and a second direction opposite from the first direction.

In an exemplary construction of handle 340, articulation wheel 312 and components of handle 340 may include hard stops indicating an articulation threshold (i.e., a maximum degree of movement or bending that the articulation wires 316, 317 can achieve). The hard stops may limit the rotation of one or more actuators coupled to articulation wheel 312 and limit the rotation of articulation wheel 312. Definitive hard stops may limit the rotational range of a knob to distinct predetermined positions and may limit movement of articulation wires 316, 317.

For example, articulation wheel 312 may be equipped with hard stops (e.g., protrusions or ridges on articulation wheel 312) which contact complementary features on handle 340 (e.g., protrusions, recesses, or grooves that receive and/or abut the protrusions or ridges on articulation wheel 312 at predefined positions). As the hard stop features of articulation wheel 312 interact with corresponding features on handle 340, this prevents articulation wheel 312 from rotating further and limits the movement of articulation wires 316, 317.

FIGS. 3A and 3B show the relative position of articulation wires 316, 317 when shaft 320 (e.g., similar to shaft 120, 220) is substantially straight and articulation wheel 312 is in a neutral position. Note that articulation wire 316 is positioned on an opposite side of shaft 320 from articulation wire 317. A neutral position may be when the actuator (e.g., actuator 203 or a knob coupled to articulation wheel 312) is not being moved or otherwise manipulated by a user, and in some examples the actuator may be biased towards the neutral position. As shown in FIG. 3A, the proximalmost ends 316A, 317B of articulation wires 316, 317 are positioned at the same longitudinal position relative to handle 340 when shaft 320 is in a substantially straight configuration and articulation wheel 312 is in a neutral position. A hard stop incorporated into handle 340 may be configured to limit the range of motion of an articulation section of shaft 320 when shaft 320 is substantially straight (shown in FIGS. 3A and 3B) by limiting the rotation of articulation wheel 312, for example by limiting the number of degrees articulation wheel 312 may be rotated clockwise or counterclockwise about rod 314.

FIGS. 4A and 4B show the relative position of each of articulation wire 316 and articulation wire 317 when shaft 320 is bent to conform to a tortuous pathway (e.g., includes several bends). As seen in FIG. 4B, shaft 320 may be bent in several directions, causing the relative position of articulation wires 316, 317 to vary. For example, articulation wire 317 may follow an inner path 370 shorter in length than an outer path 380 followed by articulation wire 316. Conversely, articulation wire 316 may follow an outer path 380 that is longer, increasing tension on articulation wire 316. As a result of articulation wire 316 being positioned along the outer path 380 in FIG. 4B, articulation wire 316 may be pulled distally to compensate for the additional length of the outer path 380 relative to the inner path 370 where articulation wire 317 is positioned. As shaft 320 bends, one of articulation wires 316, 317 may accrue slack while the other articulation wire 316, 317 is taut, such as articulation wire 317 accruing slack as a result of the inner path 370 position within shaft 320 in FIG. 4B.

The unequal tension caused by bends in shaft 320 may create an unequal position (e.g., non-neutral position) of articulation wires 316, 317 and may bias articulation wheel 312 towards a specific hard stop of articulation wheel 312. For example, when shaft 320 is positioned as shown in FIG. 4B and includes several bends, articulation wheel 312 is biased towards a hard stop limiting the clock-wise rotation of articulation wheel 312 because the bends in shaft 320 have caused articulation wheel 312 to rotate clockwise from the tension in articulation wire 316, due to articulation wire 316 following the outer path 380 of shaft 320 in FIG. 4B. This bias in articulation wheel 312 occurs without any articulation of an articulation section 150 of shaft 320. Accordingly, the position of shaft 320 in FIG. 4B (i.e., including several bends) causes articulation wheel 312 to be rotated towards a hard stop feature without achieving any articulation of articulation section 150. Thus, a user may only be able to rotate articulation wheel 312 a limited number of degrees to articulate articulation section 150 and will not achieve the same amount of articulation of articulation section 150 compared to when articulation wheel 312 is in a neutral position. Such bias in the articulation wheel 312 caused by a bent articulation shaft 320 may reduce the amount of articulation of a distal portion (e.g., an articulation section 150) of shaft 320 that can be achieved by actuating articulation wheel 312.

Slack in articulation wire 316 may prevent articulation wire 316 from becoming taut even with rotation of articulation wheel 312. When shaft 320 is bent as shown in FIG. 4B, a user may need to rotate articulation wheel 312 more than if shaft 320 was in a straight configuration (e.g. FIG. 3B) to achieve the same amount of movement/articulation of articulation section 150, due to the slack created in articulation wire 317 from the bent shaft 320. Thus, articulation wheel 312 needs to be rotated more to collect the slack in articulation wire 317 and then bend the articulation section of shaft 320. However, a hard stop of handle 340 may prevent rotation of articulation wheel 312 beyond a particular number of degrees, regardless of the position of shaft 320. The hard stop may prevent a user from rotating articulation wheel 312 the additional number of degrees needed to compensate for the slack in articulation wire 317 and to achieve a full range of motion of the articulation section of shaft 320. This will result in a limited range of motion of the articulation section of shaft 320 when shaft 320 is in the bent configuration shown in FIG. 4B. The actuators and related devices and systems discussed in this disclosure may allow a user to compensate, via an actuator, for such slack in articulation wire 317 while also maintaining full range of motion of the articulation section of shaft 320.

The below disclosure introduces an alternative to the exemplary construction of handle 340 including hard stops described above. In the alternative, at lower torque levels, a knob may operate normally by rotating rod 314 and articulation wheel 312 to move articulation wires 316, 317. As an articulation section 150 of shaft 120, 320 bends, one of articulation wires 316, 317 applies an increasing amount of torque to articulation wheel 312. Instead of including a hard stop feature in handle to stop articulation wheel 312 from rotating (such as by abutting a hard stop feature), the knob may be configured to slip relative to rod 314 when a threshold torque value is reached, such as a torque value applied to articulation wheel 312 when articulation section 150 is bent ninety or one hundred and eighty degrees. For example, a threshold torque value of 5 lbs to 15 lbs (i.e., 2.268 kg to 6.804 kg). Thus, the articulation of articulation wires 316, 317 can be limited by this “slip clutch mechanism” when the knob slips relative to rod 314 and prevents the knob from further bending articulation section 150 beyond ninety degrees, one hundred and eighty degrees, or any other maximum number of degrees bending. The maximum number of degrees bending for articulation section 150 may be set by the maximum torque value that the knob can maintain a fixed coupling to rod 314 before slipping relative to rod 314. Since this, “slip clutch mechanism” does not limit the rotation of knob by a physical hard stop set a specific number of degrees of rotation of the knob, a user may rotate the knob beyond the point where a handle with hard stops would stop. By allowing for further rotation of the knob, an articulation wire with slack (e.g., as seen in articulation wire 317 in FIGS. 4A and 4B) may be tensioned, enabling the user to achieve full articulation of the distal tip. This eliminates the problem caused by slack collecting in an articulation wire 316, 317 because of a bent shaft 320 and allows a user to articulate articulation section 150 through a full range of motion regardless of the position of shaft 320.

FIG. 5 shows a perspective view of a knob 543 configured to achieve this “slip clutch mechanism.” Knob 543 is shown as transparent to expose structural features of the knob 543, and knob 543 is shown coupled to a control rod 514. Knob 543 may include prongs 531 that assist a user in gripping knob 543. Prongs 531 may be positioned around the circumference of knob 543 to accommodate various hand sizes and gripping preferences. Knob 543 may be incorporated into any of the medical devices discussed herein (e.g., medical device 100) or any other medical device incorporating articulation wires. Knob 543 may be similar to articulation actuator 203 described above, except as described herein. For example, knob 543 may be coupled to an articulation wheel (e.g., articulation wheel 212 coupled to articulation wires 216, 217), for example through control rod 514. Knob 543 may be actuated in a first and a second direction (e.g. rotated about a central axis 599), which may cause the articulation wheel to rotate in the same first or second direction, and articulation wires may be tensioned to cause a distal end of a shaft (e.g., tip 130) to move (e.g. bend articulation section 150, etc.).

Knob 543 may include a lumen 571, which may extend entirely through a central portion of knob 543. Lumen 571 may include a first portion 536 and a second portion 538, and the first portion 536 may have a larger diameter than the second portion 538. The first portion 536 be configured to receive an end portion of rod 514, and the first portion may include a plurality of planar surfaces that may be polygonal, hexagonal, dodecagonal, or any other shape forming radially-inward facing surface 537 (e.g., an interior surface), relative to central axis 599 (e.g. a central longitudinal axis of lumen 571). The first portion 536 may be configured to correspond to and align with the circumferential shape of control rod 514. In other words, first portion 536 may have a radially inward facing surface 537 that corresponds to a radially-outward facing surface of control rod 514.

Control rod 514 may also be polygonal, hexagonal, dodecagonal, or any other suitable shape that corresponds to the shape of first portion 536 of lumen 571. For example, control rod 514 may include beveled surfaces 546 extending from a lip 544 formed in control rod 514 such that beveled surfaces 546 form a portion of control rod 514 with a smaller diameter relative to a proximal portion 515 of control rod 514. Lip 544 may be configured to abut knob 453 when control rod 514 is coupled to knob 543. Control rod 514 may be introduced and received in first portion 536 of knob 543. Once assembled, control rod 514 may be configured to receive an articulation wheel (e.g., articulation wheel 212 coupled to articulation wires 216, 217). For example, control rod 514 may extend from a portion within handle 240 where control rod 514 is coupled to an articulation wheel 212, to a portion outside of handle 240 where control rod 514 is coupled to knob 543. Control rod 514 may be rotatably coupled to handle 240.

The interaction between the shape and/or polygonal surface of control rod 514 and the shape and/or radially-inward facing surface 537 of first portion 536 of knob 543 may allow knob 543 to slip relative to control rod 514 at a threshold torque value (i.e., a specific level of torque required to trigger the engagement of the slip clutch mechanism). An exemplary torque value for the embodiments described may be between 5-15 lbs. At low torque values, knob 543 may not slip relative to control rod 514 and rotation of knob 543 may result in rotation of control rod 514. However, as torque increases, knob 543 may reach a threshold torque value where knob 543 slips relative to control rod 514 and does not rotate control rod 514 as knob 543 is rotated. When rotational force is applied to knob 543 during a procedure, and a torque threshold is reached, the interaction between the control rod 514 and first portion 536 may facilitate slipping between control rod 514 and first portion 536, to avoid over-articulation of articulation section 150 and/or avoid breaking one or more components of medical device 100, such as articulation wires 216, 217.

When actuating knob 543 and the threshold torque value of knob 543 is reached, beveled surfaces 546 of control rod 514 may slide across a radially-inward facing surface 537 of first portion 536 As slippage occurs, feedback mechanisms, such as tactile cues or device noises, may be integrated into the device's user interface to provide real-time feedback on torque levels, knob position, and whether the threshold torque value has been reached. For example, textured surfaces on radially-inward facing surface 537 may provide tactile cues to indicate an operating state, such as a first state when knob 543 is not slipping relative to control rod 514 and a second state when knob 543 is slipping relative to control rod 514.

Furthermore, the slip clutch mechanism may provide a means for a user to compensate for slack that accrues in one or more articulation wires 216, 217, 316, and 317 due to a shaft (e.g., shaft 320 in FIGS. 4A and 4B) bending through tortuous pathways. As the device navigates through tortuous pathways, the relative position of articulation wires (FIG. 4A) may vary, leading to slack on one side (e.g., interior path 370 side) and tension on the other (e.g., exterior path 380 side). By allowing the knob to slip at the threshold torque value, users may effectively manage this slack while also being prevented from over-torqueing the articulation wires. Since the knob is limited by a maximum torque value and not a maximum number of degrees of rotation, a user may rotate knob 543 as much as is needed to achieve tension (and collect slack) in an articulation wire. The knob 543 may slip at a maximum torque value to prevent over-torqueing the articulation wire and prevent potentially breaking the medical device 100. The additional rotational capability may help users achieve full articulation of the distal tip regardless of how many bends are present in shaft 120, 320.

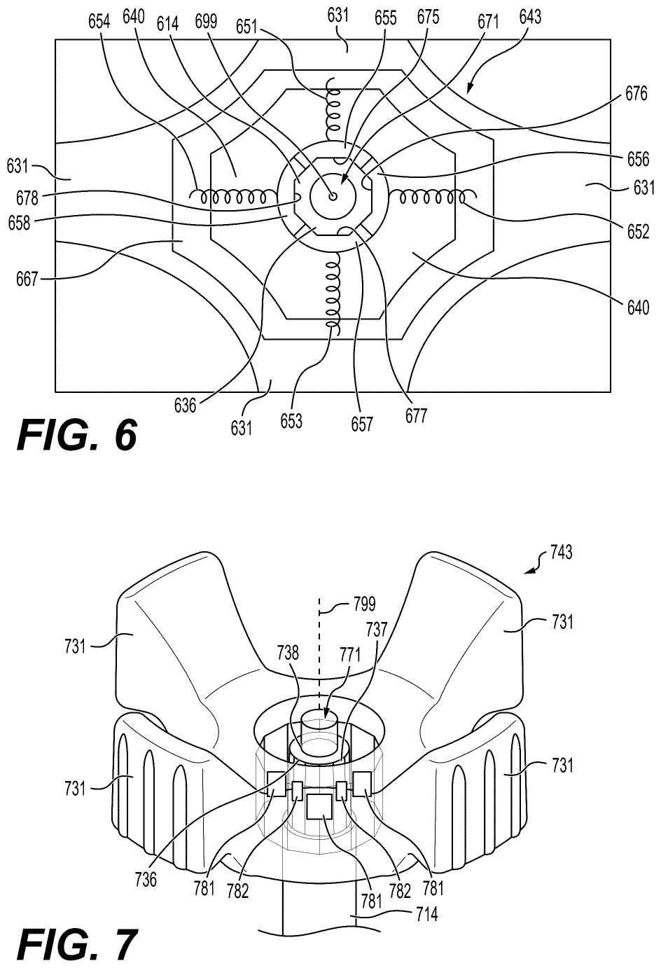

FIG. 6 shows a back view of another embodiment of a knob 643 that may be configured to achieve the slip clutch mechanism described herein. Knob 643 is shown coupled to a control rod 614, and knob 643 and control rod 614 may have any of the features described herein related to any other knob described herein (e.g., 142, 143, 203, 543, 743 and control rods 214, 314, 514, 714. Knob 643 may include a lumen 671, which may be similar to lumen 571 and may include a first portion 636 similar to first portion 536. For example, first portion 636 may be shaped to be polygonal, hexagonal, octagonal, dodecagonal, or any shape corresponding to and capable of aligning with the shape of a portion of control rod 614.

Control rod 614 may also be polygonal, hexagonal, dodecagonal, or any other suitable shape that corresponds to the shape of first portion 636 of knob 643. In FIG. 6, control rod 614 and a radially-inward facing surface of first portion 636 are both octagonal.

First portion 636 may be formed by a plurality of semicircular components 655, 656, 657, 658. Each of Semicircular components 655-658 may include a radially-inward facing portion 675, 676, 677, 678 and the radially-inward facing surfaces 675-678 of components 655-658 may form the radially-inward facing surface of first portion 636. The number and size of semicircular components 655-658 in FIG. 6 is merely exemplary. For example, the number of semicircular components 655-658 may be adjusted based on the size, shape, and other dimensions of control rod 614. In addition, the radially-inward facing surfaces 675-678 of semicircular components 655-658 may be configured to form a shape that aligns with the shape of control rod 614. Each of semicircular components 655-658 may be moveably coupled to a main body 640 of knob 643 via a spring 651-654 (e.g., a suitable biasing member), and may move relative to main body 640. Springs 651-654 will be discussed in further detail below.

Control rod 614 may be positioned in first portion 636 of knob 643 (i.e., similar to control rod 514) and receive an articulation wheel (e.g., articulation wheel 212 coupled to articulation wires 216, 217). In addition, knob 643 may include prongs 631 similar to prongs 531.

Knob 643 may include a recessed portion 667 that extends around a central axis 699 of knob 643, and recessed portion 667 may face a handle body 202 when knob 643 is coupled to handle body 202. The plurality of springs 651, 652, 653, 654 may be fixedly coupled to a portion of the recessed portion 667 and may be positioned circumferentially around central axis 699. Each of semicircular components 655-658 may be coupled to a separate spring 651-654. Each of springs 651-654 may be positioned to exert a radially-inward force, relative to central axis 699, on one of the semicircular components 655-658 when compressed, and may be configured to move semicircular components 655-658 towards a position in which semicircular components 655-658 are aligned with and abut control rod 614.

Springs 655-658 are configured to allow semicircular components 655-658 to move radially outward, relative to central axis 699, when a threshold amount of force (e.g. torque) is applied to semicircular components 655-658 through the interaction between knob 643 (e.g. semicircular components 655-658) and control rod 614 as knob 643 is rotated by a user (e.g. rotational force applied from turning knob 643). When the threshold amount of torque is reached, springs 651-654 may compress and semicircular components 655-658 may move radially outward, relative to central axis 699, to allow knob 643 to slip relative to control rod 614. Said differently, rotation of knob 643 may rotate control rod 614 until a threshold level of torque is reached, and when the threshold level of torque is exceeded semicircular components 655-658 will move radially outward and rotation of knob 643 will not result in rotation of control rod 614. The radially-outward movement of semicircular components 655-658 expands the diameter of first portion 636 such that first portion 636 is no longer fixedly coupled to control rod 614. Knob 643 may return to being fixedly coupled to control rod 614 when the torque applied to knob 643 is below the threshold level of torque. The polygonal shape of semicircular components 655-658 and control rod 614 may facilitate transitioning from a slipping state, in which rotation of knob 643 does not result in rotation of control rod 614, and a fixed state, in which rotation of knob 643 does result in rotation of control rod 614 (e.g. a state in which knob 643 is temporarily fixedly coupled to control rod 614).

The properties of springs 651-654, such as their stiffness and/or compression/expansion properties, may be tailored to adjust the threshold torque value for knob 643 as needed for different applications. Each of the plurality of springs 651-654 may extend from a wall of recessed portion 667 toward one of the plurality of semicircular components (655-658), and an end of each spring 651-654 may be fixedly coupled to a semicircular component 655-658. For example, FIG. 6 shows spring 651 may be coupled to semicircular component 655.

FIG. 7 shows another embodiment of a knob 743 that may be used to achieve the slip clutch mechanism described herein. Knob 743 is shown coupled to control rod 714, and knob 743 and control rod 714 may have any of the features described herein related to any other knobs described herein (e.g., 142, 143, 203, 543, 643 and control rods described herein (e.g., 214, 314, 514, 614). Knob 743 may include prongs 731 (i.e., similar to prongs 531) that assist a user in gripping knob 743.

Knob 743 may also include a lumen 771, which may be similar to lumen 571 and/or lumen 671. For example, lumen 771 may include a first portion 736 similar to first portions 336, 536, 636 and interior surface 737 of lumen may be shaped to be polygonal, hexagonal, octagonal, dodecagonal, or any shape corresponding to and capable of aligning with the shape of control rod 714, described below. In other words, first portion 736 may have a circumference or an interior surface 737 facing radially inward that corresponds to the shape of a control rod 714. Control rod 714 may also be polygonal, hexagonal, dodecagonal, or any other suitable shape that corresponds to the shape of lumen 771 of knob 743.

Control rod 714 may include one or more magnets 782, and knob 743 may also include a plurality of magnets 781 positioned within knob 743 and positioned circumferentially around the first portion 736 of lumen 771. Magnets 781, 782 may be fixedly coupled or removably coupled to knob 743 and control rod 714, respectively. Additionally, the number of magnets 781, 782 in FIG. 6 is merely exemplary. For example, the number of magnets 781, 782 may be adjusted based on the shape (i.e., the number of sides) of control rod 714 and knob 743, and may include any suitable number of magnets 781, 782. The embodiment of FIG. 7 may achieve a slip clutch mechanism in a similar manner to the embodiment of FIG. 5 and FIG. 6, except as described herein.

Magnets 782 may be arranged circumferentially around lumen 771 within/on knob 743, and magnets 781 may be positioned within control rod 714 and/or at a radially-outer surface of control rod 714, relative to a central longitudinal axis 799 of control rod 714. Magnets 781 may have their positive pole oriented towards the central longitudinal axis 799 of lumen 771 and their negative pole oriented away from the central longitudinal axis of lumen 771. Magnets 782 may have their negative pole oriented away from the central longitudinal axis of control rod 714/lumen 771 and their positive pole oriented towards the central longitudinal axis of control rod 714/lumen 771. Alternatively, magnets 781 may have their negative pole oriented towards the central longitudinal axis 799 of lumen 771 and their positive pole oriented away from the central longitudinal axis of lumen 771. Magnets 782 may have their positive pole oriented away from the central longitudinal axis of control rod 714/lumen 771 and their negative pole oriented towards the central longitudinal axis of control rod 714/lumen 771.

The slip clutch mechanism in the embodiment of FIG. 7 may rely on the interaction between magnets 781 positioned circumferentially around lumen 771 of knob 743 and magnets 782 on control rod 714. For example, magnets 782 on control rod 714 and magnets 781 of knob 743 may allow knob 743 to slip between several positions at a threshold torque value relative to control rod 714. Magnets 781 and magnets 782 may be configured to hold knob 743 in a fixed position relative to control rod 714, and rotation of knob 743 by a user may result in a direct drive of control rod 714 to rotate control rod 714, until a threshold value of torque is applied to knob 743. When the threshold value of torque is applied to knob 743 (e.g., by rotating knob 743 at the threshold torque value), knob 743 may slip relative to control rod 714 and the magnets 782 of knob 743 may move position relative to magnets 782 of control rod 714. This “slipping” mechanism prevents a user from actuating control rod 714 with a torque beyond the threshold value of torque for knob 743. The magnetic force of magnets 781, 782 may be configured to achieve the threshold torque value of knob 743, and this threshold torque value may be associated with a ninety-degree bend, one-hundred-and-eighty-degree bend, or any other degree of bending of an articulation section 150 of medical device 100. The position of each of magnets 782 on control rod 714 may correspond to specific positions or angles of knob 743 (i.e., if there are four magnets 782 on control rod 714, there may be a first, second, third, and fourth position corresponding to each of the four magnets 782). For examples, magnets 782 of control rod 714 may be evenly spaced circumferentially around control rod 714, and magnets 781 of knob 743 may be evenly spaced circumferentially around lumen 771.

At low torque values, knob 743 may not slip to other positions relative to control rod 714. Once the force on knob 743 reaches and/or exceeds a threshold torque value, the attraction force between magnets 781, 782 that is coupling knob 743 to control rod 714 may give way (i.e., torque, exceeds the magnetic force holding magnets 781, 782 together (e.g. proximate to each other), leading to separation of magnets 781, 782 allowing knob 743 to slip relative to the control rod 714. This slipping action enables knob 743 to transition between coupled positions (e.g., 90 degrees from each other). Additionally, any of the previously described features of any of control knobs 143, 543, 643 may be used independently or in combination with the embodiment of FIG. 7.

The medical devices, systems, and methods discussed in this disclosure may facilitate articulation of an articulation section of a medical device, may reduce the procedure time, may reduce the risk of damaging one or more medical devices during a procedure, and may reduce the risk of injury to the patient. For example, the devices in this disclosure may reduce the need to reposition a shaft of a medical device in order to achieve full articulation of an articulation section of the shaft. The devices, systems, and methods of this disclosure may prevent breaking of articulation wires during a procedure and may facilitate moving an articulation section of a shaft through a full range of motion regardless of the position of the shaft and how many bends the shaft may include.

Any of the medical devices 100, knobs 243, 343, 643, 743, described herein may be made of any suitable material, such as plastic, PEBAX, or any other suitable material. In any of the above-described examples, medical device 100 may include one or more components that are metallic, a polymer, machined, formed, stamped, insert molded, or any combination thereof. Any aspect(s) of any one of the above-described examples of knobs 143, 343, 643, 743 and other components of medical device 100 may be incorporated into any of the other examples of medical devices 100 described herein.

The integration of one or more of the knobs described herein featuring slip clutch mechanisms, may resolve one or more challenges posed by traditional hard stop designs of conventional articulation knobs. While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
a knob having a recessed first portion, wherein the knob is configured to actuate an articulation wire;
a control rod configured to be received within the first portion of the knob; and
a shaft including an articulation section, wherein actuation of the knob is configured to move the articulation section;
wherein the knob is configured to transition between (1) a first state in which the knob is fixedly coupled to the control rod and (2) a second state in which the knob is moveable relative to the control rod;
wherein the knob is configured to transition from the first state to the second state when a threshold torque value is applied to the knob.

2. The medical device of claim 1, wherein the first portion includes a radially-inward facing surface, relative to a central longitudinal axis of the first portion, that is polygonal shaped, and the control rod includes a radially-outer facing surface, relative to a central longitudinal axis of the control rod, that aligns with the polygonal shaped radially-inward facing surface of the first portion.

3. The medical device of claim 2, wherein the radially-inward facing surface includes a plurality of planar surfaces.

4. The medical device of claim 1, wherein the threshold torque value is within a range of 5 lbs to 15 lbs.

5. The medical device of claim 1, wherein the control rod is coupled to the articulation wire and the articulation wire extends longitudinally through the shaft.

6. The medical device of claim 5, wherein rotation of the knob is configured to move the articulation wire proximally or distally when the knob is in the first state, and wherein rotation of the knob is configured to not move the articulation wire proximally or distally when the knob is in the second state.

7. The medical device of claim 1, wherein the knob includes a plurality of semicircular components surrounding the control rod, wherein a radially-inward facing surface of each of the plurality of semicircular components align with a shape of the control rod.

8. The medical device of claim 7, wherein each of the semicircular components is coupled to a separate biasing member.

9. The medical device of claim 8, wherein each of the biasing members is a spring, and wherein each of the springs is configured to bias one of the plurality of semicircular components towards a central longitudinal axis of the first portion.

10. The medical device of claim 9, wherein upon reaching the threshold torque value, each of the biasing members are configured to compress and increase a diameter of the first portion.

11. The medical device of claim 10, wherein when the threshold torque value has not been reached, each of the semicircular components abuts at least two other semicircular components.

12. The medical device of claim 1, wherein the control rod includes at least one first magnet, and wherein the knob includes at least one second magnet.

13. The medical device of claim 12, wherein a positive pole of the at least one first magnet faces a direction away from a central longitudinal axis of the control rod, and wherein a negative pole of the at least one second magnet faces towards the central longitudinal axis of the control rod.

14. The medical device of claim 12, wherein the at least one second magnet includes a plurality of second magnets, and the plurality of second magnets is arranged circumferentially around the first portion of the knob.

15. A medical device, comprising:
a knob having a recessed first portion, wherein the knob is configured to actuate an articulation wire, and the first portion is configured to receive a control rod; and
wherein the knob is configured to transition between (1) a first state in which the knob is fixedly coupled to the control rod and (2) a second state in which the knob is moveable relative to the control rod;
wherein the knob is configured to transition from the first state to the second state when a threshold torque value is applied to the knob.

16. The medical device of claim 15, wherein a radially-inward facing surface, relative to a central longitudinal axis of the first portion of the knob includes a plurality of planar surfaces and corresponds to a polygonal surface of the control rod.

17. The medical device of claim 15, wherein the knob further comprises at least one magnet.

18. The medical device of claim 15, wherein the knob further comprises at least one biasing member coupled to a semicircular component of the first portion, and wherein the semicircular component is moveable relative to a plurality of prongs of the knob.

19. A medical device, comprising:
a knob having a radially-outer portion and a recessed first portion, wherein the knob is configured to actuate an articulation wire, and the first portion is configured to receive a control rod;
wherein the knob is configured to transition between (1) a first state in which the knob is fixedly coupled to the control rod and (2) a second state in which the knob is moveable relative to the control rod;
wherein the knob is configured to transition from the first state to the second state when a threshold torque value is applied to the knob; and
wherein the first portion includes a plurality of semicircular components, wherein each of the plurality of semicircular components is (i) coupled to a separate biasing member of a plurality of biasing members and (ii) moveable relative to the radially-outer portion of the knob.

20. The medical device of claim 19, wherein the plurality of semicircular components is configured to move away from a central longitudinal axis of the knob when the plurality of biasing members is compressed and moved radially outward.
